(19) United States
(12) Patent Application Publication
(10) Pub. No.: US 2002/0114846 A1
(43) Pub. Date: Aug. 22, 2002

(54) Amniotic apoptosis modulating substances

(76) Inventor: Vladimir Bakhutashvili, Tbilisi (GE)

Correspondence Address: Albert Wai-Kit Chan, Attorney at Law, Suite 604, 141-07 20th Ave., Whitestone, NY, 11357, US

(21) Appl. No.: 09/928,178

(22) Filed: Aug. 9, 2001

Related U.S. Application Data
(60) Provisional application No. 60/224,112, filed on Aug. 9, 2000.

(51) Int. Cl. A61K 35/54

(52) U.S. Cl. 424/582

(57) ABSTRACT

This invention is directed to methods of obtaining compounds from human amniotic tissue and/or by synthesizing these compounds by chemical and genetic engineering methods known in the art that modulate apoptosis in animals, including humans, their preparation, their applications in human conditions for the treatment of all disease conditions and other conditions in which apoptosis occurs and in laboratory tests for diagnostic studies and other potential uses. The invention describes methods of obtaining compositions that modulate apoptosis and compositions obtained thereby. These compositions are herein referred to as Amnion Apoptosis Modulators (AAM). AAM includes materials comprised of biologically active factors found in amniotic tissue and amniotic fluid associated therewith. AAM could be manufactured from the amniotic tissue of mammalian origin—human, pig etc. All AAMs, derived from amnions or chemically or genetically prepared are physiologically acceptable for administration in amount sufficient to modulate apoptosis. The invention encompasses methods of use of the AAMs.

Throughout this application, references are made to various publications. Disclosures of these publications in their entireties are hereby incorporated by reference into this application to more fully describe the state of the art to which this invention pertains.

### FIELD OF THE INVENTION

The invention(s) is directed to method(s) of obtaining compounds from human amniotic tissue and/or by synthesizing these compounds by chemical and genetic engineering methods known in the art that modulate apoptosis in animals, including humans, their preparation, their applications in human conditions for the treatment of all disease conditions and other conditions in which apoptosis occurs and in laboratory tests for diagnostic studies and other potential uses.

### BACKGROUND OF THE INVENTION

### Apoptosis

Apoptosis is a mode of cell death that occurs under normal physiological conditions. It is an active genetically controlled process, which removes unnecessary and damaged cells. Apoptosis enables living organisms to control cell numbers in tissues and to eliminate individual cells that jeopardize the living organism. It takes place in developing embryos and in adult organisms during physiological tissue turnover and in most pathological processes. Jacobson et al., Cell (1997) 88(3): 347-354; Kauffman-Zeh et al., Nature (1997) 385(6616):544-548; Ashkenazi et al., (1998) Science 281: 1305-1308; Dixit 1999; Thatte, Dahanukar, 1997

Apoptosis, is a Greek word that describes the process of “leaves falling from a tree” and can be understood as that naturally occurring process. It is a mode of cell death that occurs in plants and animals under normal physiological conditions as well under conditions of disease and trauma.

Characteristic morphological features of cells undergoing apoptosis include condensation of nuclei and cytoplasm, blebbing of cytoplasmic membranes and finally, fragmentation into apoptotic bodies that are phagocytosed by neighboring cells. Cohen, (1993) Immunology Today 14:136

Recent studies indicate the role of abnormally regulated apoptosis in pathogenesis of large variety of diseases such as gastrointestinal, cardio-vascular, HIV infection, rheumatoid arthritis, acute pancreatitis, etc., as well as aging and different pathological conditions related to it. Finkel T. H., et al., (1999) JAMA 282(11): 1021-2; Vocero-Akbani A. M., (1999) Nat.Med. 5: 29-33; Lovell D. J., (2000) N Eng J Med 342(11): 763-9; Shirin H., (1998) Gut 43(5): 592-4; James T. N., (1997) Circulation 96(5): 1696-700. Davies M. J., (1997) Heart 77(6): 498-501; Dewuhrst S. (2000) Frontiers in Bioscience 5, d30-49; Telford W. (1999) Cell Immunol 191:131-138

Therefore substances that are able to modulate apoptosis are applicable to correcting medical problems stemming from particular cellular excess or deficiency.

Regulation of genes involved in energy metabolism, angiogenesis, NO (nitrous oxide) metabolism and apoptosis are the main mechanisms that become activated in ischemic conditions. A key role in the activation of gene transcription is the DNA-binding complex termed “hypoxia-inducible factor-1” (HIF-1) (Ratclif P. J., et al., 1998; Wenger R. H. and Gassmann M., 1997; Blancher C. and Harris A. L., 1998).

Activated HIF-1 regulates the expression of genes involved in adaptation of higher organisms to hypoxia. On the cellular level expression of HIF leads to the reduction of proliferation and increase of apoptosis (Carmeliet P., et al., 1998).

Acute ischemic damage is basically associated with cellular necrosis. But in myocardial infarction, renal hypoxic damage, stroke, other hypoxic damage, cells which surround the area of infarction and which are usually hypoxic, die as a result of programmed cell death—apoptosis.

### Amnion

The amnion is a biological membrane which lines and envelopes the amniotic cavity; it is composed of a simple cuboidal epithelium, a basement membrane and a vascular mesenchymal layer consisting mainly of hyaluronic acid. Amniotic tissue itself inhibits inflammation ,and acts as a fibrovascular routing epithelium, recovering agent and wound healing agent.

The amnion is derived from the product of conception, the developing fertilized ovum in contrast to the placenta which is derived from the maternal uterus. Thus, products developed from the amnion are not related to the placenta or to products developed from the placenta.

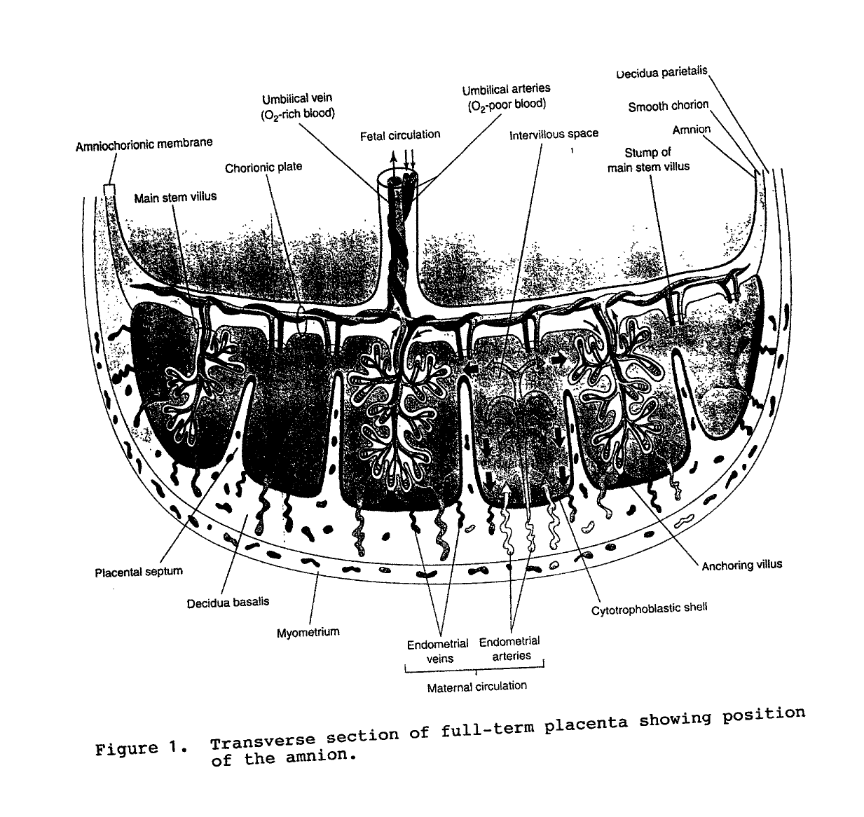

See FIG. 1 (Moore, 5^{th} edition, 1998)

Smelser G. K. Role of epithelium in incorporation of sulfate in the corneal connective tissue, in Duke-Elder s., Perkins, E. S., eds. The transparency of Cornea, Oxford: Blacwell Scientific; 1960:125, Steruli C. H. Schmidhauser C. Kobrin M, Bissell M. J. and Derynck R. Extracellular matrix regulates expression of the TGF-beta.subl gene, J. Cell Biology, 1993; 120:253-260, Dunnington J. H. Tissue responses in ocular wounds. Am J Ophtal 1957; 43:667)

### HISTORY OF DEVELOPMENT OF PRODUCTS

In 1980 Vladimir (Lado) Bakhutashvili initiated research to try to identify an inexpensive source of interferons (IF). Initially he cultured human placental tissues complete with amniotic membranes and termed his material “placental interferon, or “Plaferon.”

This pharmacologically active agent was shown to contain the following IF fractions: alpha 85-90%, beta 8-10% and gamma 3-5%. Plaferon has been tested according to IF titer in International Units (IU) and is registered as an antiviral and immunomodulatory drug by the Georgian Ministry of Health Care.

Clinical applications showed PL to possess properties similar to leukocytic IF. Early clinical studies were implemented in patients with ocular herpes and viral hepatitis. Interestingly, the effect of PL on viral hepatitis was not dependent upon its anti-viral activity. In-vitro and in-vivo studies revealed that the whole range of properties of PL such as anti-hypoxic, anti-allergic, anti-toxic or expediting of emerging from anesthesia, differed from those of other IFs. The preparation showed no species-specificity—obtained from human amnion it “worked” and was pharmacologically active in mice, rats, dogs and other experimental animals.

Based on experimental evidence that Plaferon possessed additional properties not present in interferons, a new preparation was obtained from human amnions following culture with a virus and autoclaving (heat treatment). This product was commercialized under the name PlaferonLB, (PLB). It contained no interferons yet it still possessed properties similar to Plaferon and Alpha Interferon such as anti-hypoxic, anti-allergic, anti-toxic, immuno-modulative, etc. PLB is free of HIV, B and C hepatitis viruses and prions. PLB was shown to possess clinical value to treat these conditions. Beneficial clinical observations were noted in clinical disease states when treated with PLB, but no mode of action was described and the method of production or manufacture of PLB was not described.

PLB has since been noted to have apoptosis modulating properties and is therefore considered to be an AAM. Some AAM like Plaferon also contain inerferons and therefore may have the properties of AAM and interferons.

### SUMMARY OF THE INVENTION

The present invention describes methods of obtaining compositions that modulate apoptosis and compositions obtained thereby. These compositions are herein referred to as Amnion Apoptosis Modulators (AAM). AAM includes materials comprised of biologically active factors found in amniotic tissue and amniotic fluid associated therewith. AAM could be manufactured from the amniotic tissue of mammalian origin—human, pig etc. All AAMs, derived from amnions or chemically or genetically prepared are physiologically acceptable for administration in amounts sufficient to modulate apoptosis. The invention encompasses methods of use of the AAMs.

### AAM ACTIVITY

### AAM Activity In Cultured Cardiomyosytes

Hypoxic cardiomyocytes: The ability of an AAM (Plaferon-LB, PLB) and a Fraction of PLB, P-6 to enhance survival of rat cardiomyocytes in hypoxic media was tested. Cardiomyocytes exposed to the hypoxia in vitro suffer from generalized apoptosis. At the same time cells involved in the hypoxic media in the presence of PLB showed no or very few number of apoptotic cells.

Multiple groups of spontaneously beating neonatal mouse cardiomyocytes were treated with LPS and incubated with AAMs, PLB and the Fraction termed P-6 and the observed for beating. In addition, samples of the media were used for Elisa assay for TNFalpha. AAM reduced the expression of TNFalpha.

Blood cells: Incubation of normal peripheral blood mononuclear cells with the AAM PLB during 24 hours neither stimulates nor inhibits the incidence of apoptotic cells. Mononuclear cells stimulated to proliferation by PHA also did not increase their rate of apoptic cells after incubation with AAM during 24 hours. On the other hand AAM dramatically decreases the expression of Fas (CD95) and receptor for IL-2 on the surface of lymphocytes.

Decreased expression of IL-2 receptor arrests lymphocyte proliferation which usually occurs after PHA stimulation of blood mononuclear cells; and decreased expression of Fas or “death receptor” must diminish cytotoxity of lymphocytes towards different target cells.

Blood cells: Incubation of normal peripheral blood mononuclear cells with PLB during 24 hours neither stimulates nor inhibits the incidence of apoptotic cells. Mononuclear cells stimulated to proliferation by PHA also did not increase their rate of apoptotic cells after incubation with PLB during 24 hours. On the other hand PLB dramatically decreases the expression of Fas (CD95) and receptor for IL-2 on the surface of lymphocytes (Table 1).

Decreased expression of IL-2 receptor arrests lymphocyte proliferation which usually occurs after PHA stimulation of blood mono-nuclear cells; and decreased expression of Fas or “death receptor” must diminish susceptibility of lymphocytes to the apoptotic stimuli.

TABLE 1
Influence of PLB on the resting and mitogene stimulated blood
mononuclear cell (MNC) apoptosis and different receptor expression.

| | | | | Mitogene |
|---|---|---|---|---|
| | | PLB | Mitogene | stimulated + |
| | | incubated | stimulated for | PLB |
| | Control | 24 hours | 24 hours | 24 hours |
| Apoptosis | 2.2 | 3.0 | 2.5 | 3.8 |
| (% of total | | | | |
| cells) | | | | |
| IL-2 receptor | 3.8 | 2.9 | 33.4 | 1.2 |
| (%of total | | | | |
| cells) | | | | |
| Fas (CD95) | 46.3 | 10.5 | 22.5 | 6.2 |
| (% of total | | | | |
| cells) | | | | |

The reciprocal relationship between proliferation and apoptosis is discussed in Evan and Littlewood. Apoptosis is very closely associated with growth-promoting ability of oncogenes. For example, a potent anti-apoptotic mitochondrial protein bcl-2 has growth inhibitory properties, and Ras proteins, the key transducers of mitogenic signals in normal and transformed cells trigger apoptosis. We note that many “apoptosis-proliferation regulation proteins (bcl-2, Bax etc.) located in mitochondrial membranes play direct roles in the maintenance of mitochondrial function. That fact gives us an idea of existence of a “hypoxia-apoptosis-proliferation axis.” AAM appears to act on the level of this axis.

### PHARMACOLOGICAL ACTIONS: IN VITRO

### Antiviral Activity

Plaferon, like other interferons, exhibited antiviral activity in human diploid cells inhibiting the reproduction of herpes, parotitis, rubeola and varicella viruses. The antiviral activity of Plaferon was less potent than leukocyte interferon.

### Immunomodulatory Activity

AAMs have shown dose-dependent antiproliferative activity in myeloma X-63 cells and in blast transformation reactions using human peripheral blood mononuclear cells (PBMCs) and murine splenocytes. AAM inhibited the synthesis of interleukin (IL)-1 and other growth factors but did not alter the production of IL-2 by mitogen-activated lymphocytes from healthy donors.

### Antihypoxic Activity

In vitro studies revealed that addition of an AAM to mitochondria stimulated the increased consumption of oxygen and ATP synthesis and accelerated aerobic and anaerobic glycolysis.

### Antitoxic Effects

The antitoxic effects of an AAM (PLB) were demonstrated in a study of carbontetrachloride (CCI4)-induced impairment of rat liver mitochondna. In this model, respiratory coefficients fell to minimal levels on day 4 of CCI4 injection, indicating inhibition of mitochondrial adenosine triphosphate (ATP). Treatment with an AAM (PL) however, prevented CCl4-induced decreases so that respiratory coefficients remained above 80% of control values, indicating maintenance of ATP synthesis. Liver function was also improved, further demonstrating hepato-protective properties of the agent.

### PHARMACOLOGICAL ACTIONS: IN VIVO; ANIMALS

### Antihypoxic Activity in Myocardial Infarction (Dogs, Rabbits)

The antihypoxic activity of AAMs was studied in dogs with experimental transmural myocardial infarction; 26 of 27 animals treated with an AAM (Plaferon) survived after treatment. Most of the untreated dogs died. AAM treatment prevented cardiogenic shock, fatal arrhythmia and microinfarcts. Similar results were shown with another AAM (PLB) treatment, also in dogs with their left anterior descending coronary artery ligated (Johnson et al).

AAM activity was examined in a rabbit model of adrenaline-induced cardiac injury. It was shown to protect animals from swelling and desquamination of capillary endothelial cells. This effect, in turn, inhibited aggregation of blood cells into the vessel lumen. Structure of cardiomyocytes was also preserved by treatment.

### Antihypoxic Activity in Cerebral Ischemia (Rats)

AAM was effective in an experimental model of photochemically induced cerebral ischemia in white rats. IV administration of PlaferonLB, an AAM 15 min prior to photoexcitation resulted in an 85% reduction in infarct volume, a 20% decrease in thrombic vessel density in the area of infarct and protected the brain tissue against oxygen reduction.

### Antihypoxic Activity in Renal Ischemia (Rats)

AAMs protective effects in obstructive nephropathy and renal ischemia have been evaluated. AAM treatment after urethral obstruction prevented severe tissue damage in the kidney and normal diuresis was restored after removal of the obstruction. Furthermore, treatment by AAM reversed hypertrophy in nephrectomized rats.

### Anti-inflammatory Activity of AAM

Experimental adjuvant-induced arthritis in rats is consequence of activation of Tumor Necrosis Factor (TNF) which is known as a main apoptotic stimuli in vitro and in vivo.

Rats that were injected with AAM had significantly lower manifestations of adjuvant-induced arthritis then placebo treated ones.

The following was published after the filing of the provisional patent application. Experiments show AAMs modulation of the apoptotic response of cells to different stimuli. (Bakhutashvili, A, Jaguzhinsky, L, Bakhutashvili, I, Kadagidze, D, Baryshnikov, A, Sokolovskaya, A, Zabotina, T, Bakhutashvili, V., Amnion apoptosis modulator. Int J Immunorehab 2001; 3(2): 17-22.)

Hypoxic cardiomyocytes: Ability of AAM to enhance the survival of mouse/rat cardiomyocytes in hypoxic media was tested. Cardiomyocytes exposed to hypoxia suffer from generalized apoptosis. In the presence of PLB however, hypoxic cells showed very few apoptotic cells.

Cancer cells: To test the influence of the AAM PLB on the rate of apoptosis in cancer cells the YURKAT model was used. When these cells are depleted of autocrine growth factor they undergo apoptosis. An AAM (PLB) incubated with YURKAT cells enhanced the number of these cells that underwent apoptosis in the absence of growth factor.

These two experiments demonstrate the unique ability of the AAM PLB to modulate apoptosis, increasing the incidence of cellular death in cancer cells while protecting normal cells exposed to ischemia.

### Conclusions

AAM (Plaferon and Plaferon-LB) therapy resulted in a more rapid improvement of clinical conditions in various disease states in addition to obvious improvements in laboratory indices as compared to controls. Results from experimental and clinical studies indicate that, in addition to antiviral activity, AAMs like Plaferon and PLB possess immunomodulatory, anti-hypoxic, anti-toxic and anti-allergic activities that are not characteristic of interferons. The AAMs also exhibit the following properties: anti-wrinkle, anti-inflammatory, anti-infectious, anti-viral, anti-immunogenic and anti-neoplastic. The mode of action appears to be due to apoptosis modulation.

### DETAILED DESCRIPTION OF THE FIGURES

FIG. 1. Transverse section of full-term placenta showing position of the amnion. This is a schematic drawing of a transverse section through a full-term placenta, showing (1) the relation of the villous chorion (fetal part of placenta) to the decidua basalis (maternal part of placenta); (2) the fetal placental circulation; and (3) the maternal placental circulation. (Moore, K. L. and Persaud, T. V. N., Before We Are Born: Essentials of Embryology and Birth Defects, 5th Ed., 1998, p. 128, W. B. Saunders Company)

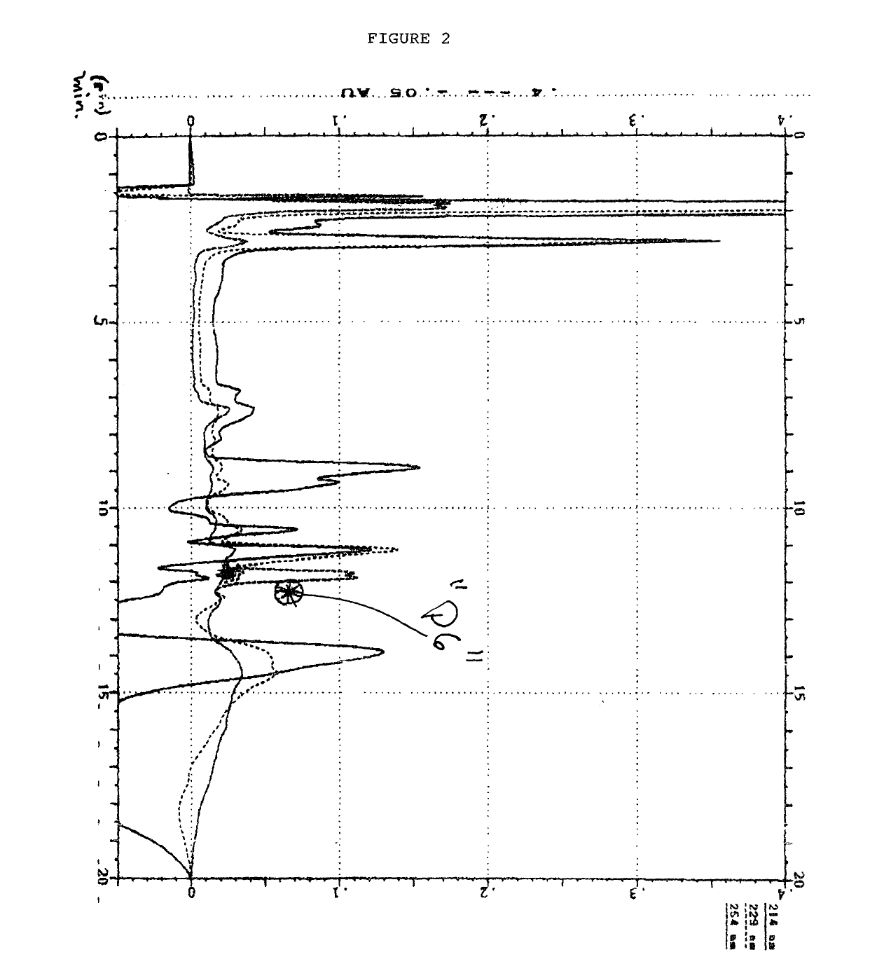

FIG. 2. Chromatography of PLB on Nova Pack C₁₈(3,9×150). Conditions: Solvent A 0,1% TFA, Solvent B 70% Acetonitrile (CAN). Flow 1,5 ml/min, gradient to 50% of B. Sample: PLB 250 μl (35 mg/ml dry powder)

P6 Purification procedure.

Dilution, MW cutting, gel-permeation and acidification.

PLB, by concentration lampoule per 1 ml is diluted in neutral distilled water—(corresponds to about 35 mg dry powder per ml). After sample is well dissolved, it is subjected to ultra filtration on 10 Kda cut-off filters and passed fraction is collected.

Obtained preparation is subjected to routine gel-permeation on Sephadex G10, for desalting. Column, parameters is depended on sample quantity applied (For an example: 20 ml of above-mentioned liquid is processed on 2I.D.X100 column) and it is recommended to monitor picks elution with appropriate UV-detector (220 nm) and collect just after voice volume eluted major pick.

After all material is processed on Sephadex G10, obtained liquid acidified by Triflouroacetic acid (TFA) 0,1-02% vol/vol

Solid phase extraction and remainder part—P6

After acidification, the liquid is subjected to solid phase extraction on cartridges containing sorbent C₁₈ (approx. characteristics: 10-15 μm, 40-100A⁰). All liquid is passed through the appropriate volume of sorbent and passed fraction is collected.

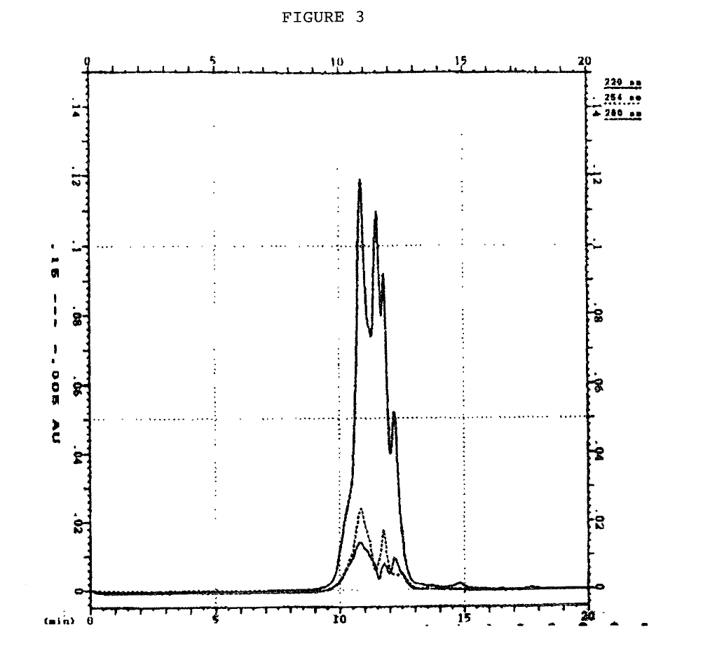

Passed fraction represents P6. It is lyophilized and after is ready for use (See FIG. 3).

FIG. 3. Chromatography of P6 on Protein-pak 60. Conditions: 0,2M Phosphate buffer, Flow rate 1 ml/min. As purification of P6 includes treatment with C₁₈ sorbent the chromatographic separation of P6 by convenient reverse phase is hard to achieve, because of extreme polarity of the constituents. Approximate MW value (estimated by calibration curve for compounds on the picture is: below 6 Kda.

### DETAILED DESCRIPTION OF THE INVENTION

### Modes of Carrying out the Invention

Besides the method described hereby compounds may also be prepared by methods known in gene engineering and peptide synthesis arts.

### Description of Type Species of Microorganisms Involved in Production

Type species “H” of Newcastle disease virus (NDV) must be utilized as an inductor of AAM.

NDV is incubated in the primary culture of chicken embryos. Absence of infectious diseases at the farm and sterility of the embryos must be verified by special veterinary certificates. Primary culture is contaminated by the virus and is incubated for 48 hours at 37° C. Then supernatant is collected and virus is stored at −20°0 C.

NDV reproduces within primary culture of chicken embryos with cytopathic activity. NDV does not reproduce in human cell cultures. The genuineness of NDV is confirmed by suppression of its cytopathic activity in the presence of specific antiserum to NDV.

Specific activity of AAM is tested in reaction of blast-transformation based on its potency to suppress proliferative activity of human mononuclear cells from the blood of healthy donors and murine splenocytes.

Specific activity of AAM is tested in reaction of blast-transformation based on its potency to suppress proliferative activity of human mononuclear cells from the blood of healthy donors and murine splenocytes.

### Requirements of Amnions

Amniotic membranes obtained from healthy mothers are used for the biosynthesis of AAM.

Blood of pregnant women is screened for syphilis, HIV and B and C hepatitis.

Results of all tests must be negative.

The placenta and all membranes (chorion and amnion) and the umbilical cord are taken from clinically healthy mothers after the normal parturition (not delayed and without premature water braking) and birth of normal baby. The tissues and the Placenta are grossly examined to be certain that they are without visual pathology, raptures, signs of atrophy and hypertrophy, in the absence of meconium. The amniotic membrane is carefully dissected free from the remaining tissues.

### TWO METHODS OF MANUFACTURING AAMs

I. Induction of AAM Biosynthesis in Amnion Tissue by Virus.

Production of AAM in the cells of amniotic membrane is inducted by NDV.

Process of production includes 5 stages: priming, induction, biosynthesis, collection of active substances and virus inactivation.

1. Priming is achieved by preliminary 2-hour incubation of amniotic tissue at 37° C. with ready AAM (0.01 mg of protein content per 1.0 ml of media). For that period of time AAM exhibits its priming-effect—as a result, amniotic cells become “alert” for the following more intense production of preparation.

2. Induction is achieved by means of NDV for 1 hour at 37° C. Within that time, virus is already absorbed at the cell surface and triggers the initiation of production of AAM.

3. Biosynthesis of AAM and cultivation of amniotic tissue takes 10-12 hours at 37° C., and AAM is extracted to culture media.

4. Liquid containing AAM is separated from amniotic tissue by centrifugation.

NDV is inactivated by reaching the pH of 2.0 in the media and incubation at +4° C. for not less than 3 days. A full inactivation is achieved for this period of time.

5. Then native AAM undergoes essential stages of purification.

### Technology Description

All laboratory glassware after thorough washing is sterilized by heat at 160° C. for 1 hour. Rubber corks are sterilized by autoclaving (Pressure-150 kPa, time-30 min).

1. Preparation of media. Media #1 is prepared under the sterile conditions: Hanks salt solution, or Media 199, or saline with addition of broad spectrum antibiotic.

2. Delivery and storage of amnion. Placentas are collected from healthy mothers after normal delivery and birth of normal child. Placentas into sterile 3 Liter glass containers with media #1. Containers with placentas are kept in hermetic thermoses and delivered by special medical transportation. After parturition placentas can be stored for no longer than 10 hours at +4° C. Each placenta is stored in separate container.

3. Primary treatment of amnion (approx. 60 min) Amniotic membrane is separated from placenta and blood clots are washed off by 200 ml of media #1. Then amnion is cut with scissors to 3×3 Cm pieces, once again treated with 200 ml of solution #1 and placed in glass container with Versen's solution preheated to 37° C. Material is being incubated for 30 min at 37° C. Then Versen's solution is being removed, amniotic tissue is cut into 0.3×0.3 Cm pieces and is washed by 100 ml of media #1. At this stage amniotic tissue is weighed.

4. Priming and viral induction. (Approx. timing—3,5 hours). Hot water bath water is preheated to 37.5° C. Under sterile conditions culture media #2 is prepared. Culture media #2 consists of 1000 ml of Media 199, Heparin—3 U/ml, donor plasma-3%, native AAM—30-40 ml/1000 ml of media, Insulin −0.0015 U/ml, Gentamycin—0.16 mg/ml.

5. Priming (2 hours): Culture media is preheated to 37.5° C. and is added in amounts of 1.0-1.5 Liters to flat-bottom flasks of chemically-proof glass. A pivot of soft iron with rustproof coating is inserted into the flask. Under sterile conditions amniotic tissue in amounts of 1 gram per 7-10 ml of media #2 is injected into the flasks, and flasks are placed at 37.5° C. water bath. Magnet mixer is placed under the water bath and the rotation of magnet is transferred to the pivot. Rotation of the pivot provides the mixing of media #2 and sustains the amniotic tissue in the state of suspension. Amniotic tissue suspension is cultivated for 2-3 hours 37.5° C. water bath.

6. Viral induction (1 hour 10 min): NDV, the inductor of AAM-genesis is added to the flask in amounts 0.3 ml of liquid with virus with titer not less than 108 TCA50/0.2 ml per 1 gram of amniotic tissue and is cultivated for at 37.5° C. water bath for 1-2 hours.

7. Separation of non-absorbed virus: Tissue suspension is poured into sterile test tubes and centrifuged for 15 minutes at G×600 at 0° C. Supernatant is collected to air-tight vessels and is autoclaved at 2 Atmospheres for 1 hour and is never used again.

### Biosynthesis of AAM (16-18 Hours)

The Composition of cultural media #3: Media 199—1000 ml, 0.005 M Na Succinate—5 ml, 0.005 M L-Glutamin—5 ml, 0.001 M CaCl2-1 m/l, Gentamycin—0.16 mg/ml.

250 ml Of media #3 is dispensed into each of four 1.6 Liter sterile flasks.

Preparation of 20% solution of HCl;

Preparation of 20% solution of NaOH;

Preparation of cultural suspension: Amniotic tissue sediment after the centrifugation is suspended in 40 ml of media #3, is divided into 4 parts, then placed in 4 sterile flasks with media #3, and cultivated in thermostat at 37.5° C. Cultivation (16-18 hours). Cultivation of amniotic tissue continues for 16-18 hours at 37.5° C., and biosynthesis of AAM is being accomplished.

Collection of liquid containing AII: Cultural liquid containing amniotic tissue is dispensed into sterile test tubes and centrifuged for 15 minutes at G×600 at 0° C. Supernatant is collected to sterile flasks and sealed with sterile rubber corks. Sediment comprising amniotic tissue and virus is collected into airtight vessels and is autoclaved at 2 Atmospheres for 1 hour.

Virus inactivation: In supernatant collected previously, pH of 2.25±0.25 is achieved by dropping of 20% solution of HCl and then material is stored for not less than 78 hours at 40 C. After that, 20% solution of NaOH is being dropped to material to achieve pH range of 7.25±0.25. Measures of pH are performed by the means of potential measurer (KP-6).

Sterility control: Ready solution is screened for viruses of B and C hepatitis and HIV.

Autoclaving of ready sterile solution: Ready solution in dispensed in amounts of 4 Liters to each of 5.0 Liter sterilization flasks, and, then is sterilized by autoclaving at 120° C. for 30 minutes. Autoclaving guaranties viral and bacterial sterility of ready solution.

### Gel Filtration

Preparation of substrate: pH of 7.2-7.5 is achieved in ready solution by addition 20% solution of NaOH, then NaCl is added to substrate to reach the concentration of 0.5 M pH range is been measured with potential measurer. The substrate in amounts of 0.5 Liters is dispensed to test tubes and centrifuged at 9000 rounds per minute for 30 minutes at 4-6° C. The supernatant is filtered first through 8-fold gauze and then through sterile membrane filter with 0.22 micron pore diameter.

The filling of column: A solution of NaCl in concentration of 1 gram/Liter is added to dry “crude” Cephadex G−25 for swelling and stored in cool dry place for 24 hours; placed into the chromatographic column (XP-9) and compressed under the flow of distilled water. Following the compression, ratio between the diameter of the gel column and its length must be not less than 1:50, that provides effective separation of low-molecular admixtures from protein fractions. About 2 Liters of preparation a cycle can be absorbed through 6 Cm thick and 300 Cm long gel column.

Sterilization of chromatographic column: Columns are filled with 5% formaldehyde dilution and stored for 24 hours at 40 C. Then column is washed with 20 Liters (10 times surmounts the volume of gel) of sterile apyrogenic water.

Passing through the chromatographic column: Protein fraction (2 Liters) is transferred to column through the system of siphons and is drained under the gauge pressure not more than 50 Cm H2O. The output of protein fraction is registered by flow densimeter. A degree of salt removing is controlled in protein fraction in a 2 ml sample: addition of 3 to 5 drops of 1M NaOH shouldn't change the color of sample to rose.

At the exit the protein fraction under sterile conditions is collected to separate glass container.

After that through system of syphones 0.1M solution of phosphate buffer at pH range of 7.2 is introduced in volume of which comprises 0.1 of the total volume of system. NaCl is added till the final 0.9% concentration of salt.

Column regeneration: Low protein compounds should be washed from the column by passage of sterile distilled water in amounts 10 times surmounting the overall amount of gel.

Sterilization by filtration: Preparation collected after the gel filtration is sterilized by passage trough a system of membranes equipped with pre-filters (with pore diameter 0.6-0.45 micron) and sterilizing filter (0.22 micron) (Millipore, USA) with 293 millimeter holder. Yield obtained from each amnion is 900-1050 ml.

The preparation of ampules: Ampules are washed once with hot water and two times with distilled water under the vacuum. Then washed ampules are placed in cassettes and dried at 150° C. for 3 hours. Dry ampules are stored vertically in special containers and sterilized by dry heat at 180° C. for 3 hours.

The preparation of distributor: All connecting parts and distributor are sterilized in autoclave by the method of humid sterilization under the pressure of 2 Atmospheres for 30 minutes. Sterile connecting parts and distributor are installed on distributing setup under sterile conditions.

Dispensing: Liquid AAM is dispensed into sterile containers.

### Lyophilisation

Freezing of preparation: Containers containing liquid AAM are covered with sterile cotton swabs, stored vertically and placed in freezer. Every container is equipped with temperature gauge for the control of temperature. The freezing of liquid AAM is achieved for 25±5 hours at −35±50 C. The cassettes are swiftly transferred to sublimation chamber. The temperature of shelves in chamber must be −10±50 C. upper and lower shelves should be equipped with temperature gauges connected to temperature register device.

Eutectic point of AAM should be −35±50 C.

Lyophilization per se: Lyophilization is performed in sublimation chamber for 44±2 hrs. Before the procedure temperature must be checked, it shouldn't excess −37±20° C.

Sublimation is initiated when the temperature of condenser reaches −45±50° C. Within the first hour residual pressure should be 10⁻²-10⁻³ mm Hg. From the beginning of the first hour and in the beginning of every following hour, gauges of lagometer controlling the heating of shelves should be monitored. The speed of shelf temperature rise is 4±2° C. an hour. Around third hour of lyophilisation temperature should be approximately 0° C., and beginning from seventh hour reaches 22±2° C. and should be maintained as such for 14 hours. Beginning from fourteenth hour temperature should be raised to 25±5° C. and maintained at that level till the end of lyophilisation. In the process of drying, the temperature of AAM is controlled by a probe inserted into one of containers. Temperature should rise not faster than 1° C. per hour. Temperature should rise above 0° C. not earlier than in 28 hours since the beginning of sublimation, should reach 30±2° C. at the top of 35 hours and should be maintained within that range for consequent 9±1 hours. Residual pressure in while sublimation chamber reaches 5×10⁻³ mm Hg, and in the end of the process shouldn't exceed 4×10⁻³ mm Hg.

Unloading of dry AAM. Before the unloading of lyophilised preparation pressure in the chamber should be raised by passing of the air dried in a column with silicone gel.

Ampule sealing. Immediately after the drying containers with AAM should be hermetically sealed.

Preparation control. Preparation is checked after the process of drying in lab of biological control. Store at temperature not more than 10° C. Expiration—2 years.

### Formulas of Solutions

### Solution 1

Hanks salt solution with antibiotic, gentamycin 0.16 mg/ml.

### Solution 2

Media 199—1000 ml—this is a widely known commercially available mixture of salts and amino acids used to culture different cells and tissues.

Heparin—3 U/ml

Human plasma—3%

AAM—30 ml/L.

Insulin—0,0015 U/ml

Gentamicin—0,16 mg/ml

### Solution 3

Media 199—1000 ml

Sodium succinate—0,005 M

Human plasma—2,5%

L-glutamin—0,005 M

CaCl₂—0,001 M

Gentamicin—0,16 mg/ml

II. Extraction of AAM From Amniotic Tissue by Chemical Agent

1. As described above, amnions are separated from Placentas and are collected from healthy mothers after normal delivery and birth of normal child. Amnions are placed into sterile 3 Liter glass containers with media #1. Containers with amnions are kept in hermetic thermoses and delivered by special medical transportation. After parturition placentas with membranes attached; (amnions) can be stored for no longer than 10 hours at +4° C. Each placenta is stored in separate container.

2. Amniotic membrane is separated from placenta and blood clots are washed off by 200 ml of media #1. Then amnion is cut with scissors to 0,2-0,3 Cm pieces, once again treated with 200 ml of solution #1 and placed in glass container with Versen's solution preheated to 37° C. Material is being incubated for 30 min at 37° C. Then Versen's solution is being removed, amniotic tissue is cut into 0.3×0.3 Cm pieces and is washed by 100 ml of media #1. At this stage amniotic tissue is weighed and tissue is suspended—1 g of tissue in 5 ml of Hanks solution.

3. Prepared suspension of amniotic tissue are boiled during 15 min at 100° C. After boiling trichlorfluoric acid (end concentration 0,1%) is added and tissue was incubated in acid during 30 min at room temperature. After incubation in hydrofluoric acid amnion tissue is precipitated by centrifugation (15 g) and supernatant containing AAM is collected and dispensed in sterile containers.

4. AAM containing supernatant is lyophilised as described above. Protein content is measured by Lowry method in lyophilised AAM.

### AAM Application Fields

As various AAMs were developed, some were used clinically to treat medical conditions. The mode of action of the first preparation Plaferon was recognized to be due in part to interferons. Other modes of actions and properties, however, remained to be determined as newer preparations like PLB and P-6 were developed, and newer and improved methods of manufacture were developed. None of these latter modes of action or methods of manufacture has been previously been described.

It is believed that the multi-potent therapeutic activity of AAMs is a result of apoptosis modulating properties.

### Cosmetic Uses

AAM preparations have been shown in unpublished experiments to have an anti-wrinkle and anti-blemish healing property when applied to the skin. The AAM is added to known cosmetic formulas for regular use to improve the appearance of wrinkles and damaged skin, including the care of striae gravidarum, scar tissue and puffy eyebags.

### Acute Viral Hepatitis B

When an AAM (Plaferon, 3000-12000 IU I/M b.i.d.) was added to standard therapy in patients with acute viral hepatitis B, clinical symptoms of the disease were more rapidly reversed. Treatment with Plaferon also resulted in normalization of biochemical parameters of liver function and a more rapid recovery from symptoms compared to untreated patients, with no toxicity. Follow-up at 12 months showed that none of the Plaferon-treated patients had relapsed. Plaferon also resulted in a 1.7-fold reduction in HBsAg-antigenemia at the time of discharge from the hospital.

Results from investigations showed that 32 patients hospitalized with acute hepatitis B had a decrease in CD4+ and an increase in CDB+ T cells as compared to healthy donors. However, T-cell immunity was restored to normal after 1 month of treatment with Plaferon LB added to standard therapy. In the control group, the number of T suppressor/cytotoxic cells returned to normal, although the reduction in T helper/inductor cells persisted.

In a randomized study in 280 patients, treatment with an AAM (PLB) produced significant changes in cellular immunity as demonstrated by a decrease in CD3+, CD4+, CD22+ and CD16+ T cells and an increase in CD8+ phenotypes. Significant positive changes were also observed in Hbe-antigenemia and seroconversion. Treatment with Plaferon LB also resulted in improvements in clinical symptoms, correction of the biochemical parameters of liver function and immunologic indices and prevented recurrence of the disease.

### Herpes Zoster Ganglioneuritis

Results from a study in which 22 HIV-negative intravenous drug users with herpes zoster ganglioneuritis were given either Plaferon injections (10,000 IU b.i.d.) or oral prednisolone (70 mg/day) for 15 days showed that Plaferon-treated patients displayed normal CD3+, CD4+ and CD8+ cell counts and improvements in neurological symptoms as compared to the prednisolone group. None of the Plaferon-treated patients experienced post-therapeutic neuralgia in contrast to 4/10 in the control group. A similar study in 36 patients with herpes zoster ganglioneuritis showed that Plaferon (10,000 IU b.i.d. for 7 days) significantly normalized the number of T cells carrying HLA-DR antigens as compared to steroid-treated controls; neurological symptoms were also improved with Plaferon treatment.

### Diabetic Peripheral Polyneuropathy

Clinical improvement of diabetic peripheral polyneuropathy was observed with Plaferon LB in a study in which 21 patients were administered the agent after correcting for carbohydrate metabolism. Normalization of electrophysiological data was also observed. Prior to treatment, patients exhibited decreases in the total number of T lymphocytes and in the ratio of T helper/inductor cells. However, patients treated for 1 month with Plaferon LB showed normal levels of CD3+ and CD4+ T-cell phenotypes as compared to controls.

### Nephropathy

Plaferon in combination with prednisolone resulted in earlier and prolonged clinical laboratory remission in children with idiopathic nephropathy syndrome (INS). In the control group, 13/40 patients had experienced acute exacerbation of the disease after 1 year as compared to only 4/50 patients in the Plaferon group. Plaferon treatment also corrected the reduction in CD3+ and CDB+ T lymphocytes observed in patients with INS prior to treatment.

### Juvenile Rheumatoid Arthritis

The effect of Plaferon in 25 patients with juvenile rheumatoid arthritis (aged 18 months to 15 years) was reported in a study in which the agent was given intramuscularly or intravenously in combination with standard therapy for 7-10 days. Treatment was well tolerated with no adverse effects. Improvements in clinical symptoms and laboratory indices, stimulation of leukocyte interferon-genesis and a trend toward normalization of humoral and cellular immunity were observed after 1 month of treatment.

### Bronchial Asthma

Plaferon was shown to be a potential alternative to steroid therapy for chronic, stable, nonatopic, steroid-resistant (i.e., non-compliant to 24 mg/day or more dexamethasone) bronchial asthma in a 24-week, double-blind, placebo-controlled, randomized study in 67 patients. Plaferon LB significantly reduced the average daily dose of oral steroid required for relief and spirometric parameters were moderately improved as compared to placebo. Accompanying in vitro studies showed that Plaferon-treated PHA-activated PBMCs displayed an increased sensitivity to dexamethasone.

### Pediatric Patients with Respiratory Infections

Plaferon LB was effective and well tolerated in 2 studies of pediatric patients with respiratory infections. In the first study, 40 children with recurrent respiratory tract infections (>6 infections/year) were treated with Plaferon LB or placebo. Immunological indices improved and the frequency of infections decreased in the Plaferon LB group (22). Similar results were obtained in the second study in which Plaferon LB was administered via aerosol inhalation to 40 infants with acute viral infections of the lower respiratory tract and compared to 30 infants given standard treatment. Clinical recovery with normalization of T-cell populations (increased CD3+ and CD4+ T cells and decrease CD8+ T cells) occurred sooner in the Plaferon LB group.

### Acute Allergic Reactions

The continuous and extended use of anticonvulsants in people with epilepsy often leads to the development of adverse reactions including acute allergic reactions and acute and chronic drug toxicity. Due to its antihistamine and antitoxic properties, Plaferon was shown to be effective against acute allergic reactions and mild toxicity associated with anticonvulsive therapy. Allergic reactions disappeared in 7/9 patients after Plaferon monotherapy and in 1 patient treated with a combination of Plaferon and antihistamine. Plaferon treatment increased recovery time from symptoms of mild acute toxicity (nausea, vomiting, headache, dizziness) in 3 patients and Plaferon monotherapy was effective in 15/22 patients with severe acute toxicity. In 4 patients in whom Plaferon was combined with general antitoxic treatment, rapid decreases in toxicity were noted. Plaferon was ineffective against drug toxicity in 3 patients in whom a change in the anticonvulsant regimen was required. The agent was slightly less effective in chronic toxicity where clinical symptoms of intoxication disappeared in 6/11 patients, with significant reductions observed in 2. Plaferon not only reduced clinical signs of drug toxicity in 36.6% of the patients but also suppressed drug toxicity as seen on EEG. The inhibition of toxicity by Plaferon enabled anticonvulsant doses to be increased to levels sufficient for achieving good clinical effects.

### Early Breast Cancer

Preliminary results reported from a study involving 8 patients with early breast cancer demonstrated that an AAM (Plaferon 90,0000 IU I/M.), given preoperatively, may be a potential immunomodulator in this disease. Poor and moderate pathological responses were observed in 3 and 4 patients, respectively; there was 1 case of severe pathology in tumor and lymph nodes. Moreover, the increased levels of the tumor serum marker, CA15.3, were normalized and increases in tumor infiltrating CD5′ T cells and CD11 macrophages were observed with Plaferon treatment.

### Psoriasis

79 patients with different forms of psoriasis have been treated with I/M Plaferon. Preparation significantly improved their clinical symptoms. Best results in this study have been achieved in patients with psoriatic arthropathy. Study revealed increased activity of immunoregulatory lymphocytes and rose in percentage of CD3+ and CD8+.

It is believed that the multi-potent therapeutic activity of PLB is a result of its anti-ischemic and apoptosis modulating properties.

### Cardiovascular Diseases

On the basis of the benefit in animals whose hearts have been rendered ischemic AAMs should have benefit in treating atherososclerotic and other types of vascular obstruction that cause ischemia of tissues, including ischemic myocardium in humans. It is reasonable to expect that AAMs will also limit myocardial cell death due to other causes such as viral and immunogenic myocardiopathies and the rejection reaction that follows transplantation. These benefits can be expected to apply to such injuries of any and all of the body organs—liver, kidney, brain, etc.

### Methods of AAM Application

AAM can be administered alone or in combination with other pharmaceutically effective agents.

Methods of administration can be topical, parenteral, gastrointestinal, transbronchial, trans alveolar and sublingual. Topical application is achieved by topical application of an ointment, cream, rinse, serum, gel, etc. containing therapeutically effective amounts of AAM. Parenteral methods of administration include, but are not limited to direct injection such as intravenous, intramuscular, or subcutaneous injections. Gastro-intestinal routs of administration include, but are not limited to, ingestion and rectal. Sublingual rout of administration, if necessary, implies dropping of solution containing therapeutically active amounts of AAM under the tongue and keeping it till absorbed. Transbronchial and trans alveolar routs of administration include, but are not limited to, inhalation, either via the mouth or intranasally and direct injection into an airway, such as through a tracheotomy. AAM can be administered not alone, but in admixture with topical cosmetically or pharmaceutically acceptable carrier.

“Topical pharmaceutically acceptable carrier” can be any substantially non-toxic vehicle conventionally employed for local administration of pharmaceuticals in which AAM will remain stable and bioavailable when applied directly to skin or mucous membranes. AAM can be dissolved in a liquid, dispersed or emulsified in a medium in a conventional manner to form a liquid preparation or mixed with a semi-solid (gel) or solid vehicle to form a paste, powder, ointment, cream, lotion, serum, rinse, etc.

Suitable topical pharmaceutically acceptable carriers include Vaseline®, petrolatum, lanoline, mineral oil, vegetable oil, animal oil, organic and inorganic waxes, like paraffin and ozocerite wax.

Admixtures can contain vitamins A, or C, E, amino acids, etc.

“Topical cosmetically acceptable carrier” can be any substantially non-toxic vehicle conventionally employed for local administration of cosmetics in which AAM will remain stable and bioavailable when applied directly to skin surface. Such vehicles are known to those in skill of the art and include, but are not limited to, cosmetically acceptable liquids, serums, creams, oils, lotions, ointments, gels, or solids, such as night creams, foundation creams, suntan lotions, sunscreens, hand lotions, or the like.

The topical compositions are administered by applying a layer to the skin or mucous membrane desired to be treated.

### P6 Purification Procedure

Fraction P6 is purified from Plaferon LB (PLB). Some of PLB's characteristics which were taken into account in the process of P6 purification are briefly described. According to our preliminary investigations PLB is quite heterogeneous and salty (NaCl), containing a mixture of compounds. It includes proteins and peptides (about 5-10% of dry powder and the protein content varies among lots or series. Up to 10 major proteins (including Albumin) in molecular range from 10 to 70 Kda were identified. Also, there was extremely low quantity but quite large diversity of low molecular weight components, as it is likely, mostly peptides are below 10 Kda. (See FIG. 2.) A biologically active fraction purification procedure from the above-mentioned preparation, which is based on results of testing in various disease/disturbance models, has been elaborated.

### Summary of Purification for Fraction P6

### Plaferon LB (“PLB”)

### sample Onc. 1-3 mg/ml Protein, pH Neutral

### Cascade Ultra- or Dia-filtration (Filters 30 and 10 Kda NMWL)

| | | |
|---|---|---|
| Protein | Protein | |
| Fraction #I | Fraction #II | Protein Fraction #III |
| >30 Kda | <30 >10 Kda | <10 Kda |

↓
Protein Fraction III
Gel permeation
chromatography on Sephadex
G10 (mobile phase water)
recovering fraction with
Ve/Vo = 1.2 to 1.8
↓
Solid phase
extraction/chromatography
on reversed phase column
(C-18, pH 2.8) recovering
fraction not retained on
the column oreluted with
water.
↓
P6

### AAM Bioassay

The biological activity of AAM was tested by ability of mixture to inhibit proliferation of peripheral blood mononuclear cells (PBMC) stimulated by mitogene Concanovaline A (Con A). Briefly, PBMC were isolated from human heparinized blood and resolved in culture media in concentration 2 000 000 in 1 ml. Suspension of PBMC was dispensed in standard 48 wells plate 0.2 ml in each. Experimental design is shown on Table 2.

TABLE 2
Experimental design of AAM bioassay.

| Well | Well | Well | Well | Well | Well | Well | Well | Well | Well | Well | Well |
|---|---|---|---|---|---|---|---|---|---|---|---|
| No 1 | No 2 | No 3 | No 4 | No 5 | No 6 | No 7 | No 8 | No 9 | No 10 | No 11 | No 12 |

Wells 1, 2, 3 - control No 1 (PBMC)
Wells 4, 5, 6 - control No 2 (PBMC + Con A)
Wells 7, 8, 9 - experiment (PBMC + Con A + AAM 0.5 mg/ml)

The plate was cultured in the incubator at the 37° C. and 100% humidity during 72 hours. After 48 hours of incubation [H³]-timidine isotope was added to each well for the measuring of the proliferation rate.

0.5 mg/ml of AAM (wells 7,8,9) must inhibit proliferation of Con A stimulated PBMC (wells 4,5,6) not less then for 50%.

### PLB Effect on H1-histamine Receptors (unpublished)

Anti-histamine activity of PLB, was investigated on culture of synaptic membranes, isolated from rat brain cortex. Different concentrations of AAM (0.1-10 nM) were added in the culture media, containing 5 nM 3H-pyrilamine. In the parallel cultures non-radioactive pyrilamine was used for detection of non-specific binding. Difference between common bound radioactivity (incubation without non-radioactive pyrilamine) and non-specifically bound radioactivity (incubation with non-radioactive pyrilamine) was considered as a specific binding of 3H-pyrilamine. Study showed that PLB did not change the affinity of ligand to its own receptor (dissociation constant in presence and in absence of PLB did not change), but reduced the number of binding sites for ligand on rat brain synaptic membranes (value of Bmax in the presence of PLB was significantly reduced).

In summary, PLB contains compounds antagonistic to H1-histaminic receptor. These findings have been confirmed by corresponding clinical study. (Bakhutashvili V., et al., 1999)

Inhibition Of Secretory Phospholipase A2 Activity (Maisuradze, E., et. al., 1998)

Impact of PLB on secretory PHOSPHOLIPASE A2 (PLA2) activity was assayed by the <<pH-stat >> method after 30 minutes of incubation at pH 8.0 and 370 C, in 16.6 mM Tris-HCl, 4.5 mM CaCl2, 0.33 mM EDTA-Na2 and 1 mg of bovine serum albumin.

Dried whole bee venom from “Sigma” and egg yolk 1-alpha-lecitin (1-alpha-phosphatidylcholine) from “Serva” were used as the enzyme and substrate sources respectively. Contents of PLB standard ampules were dissolved in 1 ml of tris—HCI and used as an PLA2 activated affector (preincubation time—15 min at room temperature). By adding various substrate concentrates to the reaction mixture, the effect of bee venom PLA2 was evaluated in the presence and absence of PLB (standard dose). The results were plotted as 1/[S] vs 1/[V].

PLB is a potent inhibitor of secretory PLA2 in vitro because it inhibited bee venom PLA2 enzymatic activity almost completely after its application in standard therapeutic dose. PLB caused time-dependent, marked (over 90%) decrease of PLA2 activity. This inhibition was significant even after 100-fold dilution of PLB (Table 3).

TABLE 3
Effect of PLB dilutions on activity inhibition of bee venom PLA2.
PLB dilutionPLA2 activity inhibition

| PLB dilutions | PLA2 activity inhibition (%) |
|---|---|
| 1:1 | 95.2 ± 2.4 |
| 1:5 | 67.8 ± 1.9 |
| 1:10 | 34.6 ± 1.4 |
| 1:50 | 24.4 ± 1.6 |
| 1.100 | 19.9 ± 1.0 |
| 1:1000 | 3.4 ± 11.2 |

Note:
each value represents a mean ± SE of six experiments performed in triplicate.

The inhibition of the enzyme was of noncompetitive nature and reached the maximum within first 10-12 min after application of compound; thus PLB decreased Vmax about 5-fold, while the Km value remained unaltered in both cases. Analysis of dose-dependent enzyme activity showed half-maximal supression (IC50) after PLB application at concentration of 0.08 mg/ml (dry weight /volume—W/v). Lineweaver—Burk plot showed that reaction catalized by bee venom PLA2 was reduced by PLB via noncompetetive inhibition reaction, in which inhibitor binds to a site on the enzyme different from the catalytic site.

### Amnion Apoptosis Modulator (“PLB”)

Amnion Apoptosis Modulator (AAM/PLB) is a multipotent natural mixture of peptides, which possesses antiischemic properties. In this work we report the effect of AAM on cellular glycolysis in aerobic and nonaerobic (ischemic) conditions

### Hypoxia; AAM Activates Glycolysis

Oxygen plays a basic metabolic role in the function of all living organisms on the earth. Mammalian cells' well-being is dependent on the fluctuations in oxygen levels and deal with hypoxia by a variety of responses on the different levels (organism, tissue and cellular). Cells may switch their metabolism to glycolysis (an anaerobic process) to decrease oxygen requirement. In tissues one may observe a local activation of nitric oxide (NO) synthesis which promotes blood flow to areas experiencing a high demand for oxygen. Finally, on the level of the organism acceleration of heart rate and lung ventilation occurs. (for review Guillemin K. and Krasnow M. A. 1997).

Regulation of genes involved in energy metabolism, angiogenesis, NO metabolism and apoptosis are the main mechanisms that become activated in ischemic conditions. A key role in the activation of gene transcription is the DNA-binding complex termed “hypoxia-inducible factor” (HIF) (Ratclif P. J., et al 1998; Wenger RH. and Gassmann M., 1997; Blancher C. and Harris AL, 1998). Activated HIF regulates the expression of genes involved in adaptation of higher organisms to hypoxia. On the cellular level, expression of HIF leads to the reduction of proliferation and increase of apoptosis (Carmeliet P., et al, 1998.).

HIF 1 and HIF 2 proteins (former predominantly expressed in endothelial cells) belong to the basic-helix-loop-helix family of transcription factors period (Per) and single-minded (Sim)—Drosophila melanogaster proteins, and mammalian aryl hydro-carbon receptor (AHR), Aryl hydrocarbon receptor nuclear translocator (ARNT) and others, which all share 150 amino acid domain PAS (Per-ARNT-AHR-Sim) (Wang J. L. et al. 1995). The target genes of HIF1 and 2 are described in Table 4.

TABLE 4
Genes under the HIF1 and HIF2 regulation.¹

| | | Direction |
|---|---|---|
| | | of |
| Function | Gene | regulation |
| Hypoxia inducible factor 1 | | |
| Glycolysis | Lactate dehydrogenase A | ↑ |
| | Phosphoglycerate kinase 1 | ↑ |
| | Aldolase A and C | ↑ |
| | Phpsphofructokinase L and C | ↑ |
| | Pyruvate kinase M | ↑ |
| | Enilase A | ↑ |
| Glucose transporters | GLUT1 | ↑ |
| | GLUT3 | ↑ |
| | GLUT2 | ↓ |
| Gluconeogenesis | Phosphoenolpyruvate | ↓ |
| | carboxykinase | |
| Energy metabolism | Adenilate kinase 3 | ↑ |
| Growth factors | Vascular endothelial growth | ↑ |
| | factor (VEGF) | |
| | Transforming growth factor β | ↑ |
| | (TGFβ) | |
| | Platelet-derived growth factor β | ↑ |
| | (PDGFβ) | |
| | Placental growth factor (PGF) | ↓ |
| | Erythropoietin | ↑ |
| Receptor expression | Flt-1 (VGEF receptor) | ↑ |
| Haem metabolism | Haem oxygenase | ↑ |
| Vasomotor regulation | Inducible nitric oxide synthase | ↑ |
| | (iNOS) | |
| | Endothelin 1 | ↑ |
| Hypoxia inducible factor 2 | | |
| Growth factors | Vascular endothelial growth | ↑ |
| | factor (VEGF) | |
| Catecholamine synthesis | Tyrosine hydroxylase | ↑ |
| Receptor expression | Tie-1 (tirosine kinase receptor) | ↑ |

¹Modified from JM Gleadle and PJRatclif (1998) Hypoxia and the regulation of gene expiession Molecular Medicine Today, March, 122-129. and C Michiels, T Arnould and J Remacle (2000) Endothelial cell responses to hypoxia: initiation of a cascade of cellular interactions. Biochimica et Biophisica Acta, 1497, 1-10.

It was shown also that hypoxia induces the activation of NFKB transcription factor which induces the expression of genes involved in immune responses, stress responses, cell growth and cell survival (Yan, S. F., et al., 1995; Schmedtje, J. F., et al., 1997).

Table 4 demonstrates the activation of glycolytic enzymes under the hypoxia-induced factors. In this connection we investigated the AAM action on glycolysis. In our studies we established a model of complete hypoxia in cell cultures. The culture medium was blown out by argon to deplete it of O₂. The content of nutrients in culture media was impoverished to repress the rate of basal glycolysis in some experiments. When porcine embryonic epithelial cells (PEEC) were cultured in described media (anoxia) almost all mitochondria of these cells did not stain by rhodamine fluorescence. The former means that almost all mitochondria lost _{Δ}Ψ and accordingly ability to concentrate fluorescent rhodamine in the mitochondrial matrix. When 0,2 mg/ml of AMM preparation was added to the PEEC cultured in anoxia rhodamine fluorescence of mitochondrial matrix was detected in noticeably larger quantities of cells then in control cultures. The mitochondrial _{Δ}Ψ in anoxia demonstrate appearance of the exogenous ATP synthesized by activation of anaerobic glycolysis.

TABLE 5
Concentration of lactate(relative units) in culture media with or
without AAM

| Exp | Culture time | Concentration of | Concentration of |
|---|---|---|---|
| No | Hours | lactate without AAM | lactate with AAM |
| 1 | 0 | 6 | 11 |
| | 0.5 | 10 | 11 |
| | 1.0 | 10 | 10 |
| | 2.5 | 50 | 90 |
| | 4.0 | 80 | 100 |
| 2 | 0 | — | — |
| | 4 | 83 | 100 |
| 3 | 0 | — | — |
| | 6 | 60 | 150 |
| 4 | 0 | — | — |
| | 20 | 80 | 111 |
| 5 | 0 | — | — |
| | 23 | 20 | 40 |

In aerobic conditions we also found the ability of AAM to stimulate glycolysis. We examined the concentration of lactate in the culture media of PEEC under aerobic conditions (Table 5).

As shown in Table 5, AAM increased the rate and amount of lactate in culture in culture media of cells after 2.5 hours of incubation. Thus AAM increased aerobic glycolysis in PEEC.

### Apoptosis suppressed by AMM Under Hypoxia.

Acute ischemic damage is basically associated with cellular necrosis. But in myocardial infarction, renal hypoxic damage, stroke, other hypoxic damage, cells which surround the area of infarction and which are usually hypoxic, die as a result of programmed cell death—apoptosis. Apoptosis is an active genetically controlled process, which removes unrequired and damaged cells. It enables the whole organism to control cell number in tissues and to eliminate individual cells that threaten the animal's survival. (Steller, H., 1995; Jacobson, M. D., et al., 1997) Apoptosis take places in the developing embryo and in the adult organism during physiological tissue turnover and in most pathological processes. (Thatte, S. & Dahanukar, S., 1997; Asukenazi, A., Dixit, V. M., 1999).

The ability of AAM to enhance survival of rat cardiomyocytes in hypoxic media was tested. Cardiomyocytes exposed to the hypoxia suffer from generalized apoptosis. At the same time cells involved in the hypoxic media in the presence of AAM showed none or very few number of apoptotic cells.

### AAM Stimulates Apoptosis Under Aerobic Conditions

In normoxia the influence of AAM on the rate of apoptosis in cancer cells was tested; we used the model of Jurkat cells (human lymphoblastoid line T-cell). When these cells are depleted from autocrine growth factor they undergo apoptosis. AAM incubated with Jurkat cells enhanced the number of cells entered to the apoptosis in the absence of growth factor (Table 6).

The incubation of normal peripheral blood mononuclear cells with AAM under normoxia during 24 hours do not stimulate or inhibit number of cells promoted to apoptosis. Mononuclear cells stimulate to proliferation by PHA also did not increase the rate of apoptotic cells after incubation with AAM during 24 hours. This data demonstrates that AAM is not toxic for normal blood cells under the normoxia.

TABLE 6
Influence of AAM on the apoptosis of Jurkat cells. Apoptosis was
assessed with the cytofluorometric analysis of hypodiploid DNA
labeled with propidium iodide (Nicoletti I.1991)

| | Incubation during 24 hours | | Incubation during 48 hours | |
|---|---|---|---|---|
| | Control | AAM | Control | AAM |
| % of | 11.38 ± 0.16 | 28.8 ± 0.83* | 7.97 ± 0.34 | 42.07 ± 0.92* |
| apoptotic | | | | |
| cells | | | | |

p < 0.05

On the other hand AAM dramatically decreases expression of Fas (CD95) and receptor for IL-2 on the surface of lymphocytes (Table 7). Decreased expression of IL-2 receptor arrests lymphocyte proliferation which usually occurs after PHA stimulation of blood mononuclear cells.

TABLE 7
Influence of AAM on the resting and mitogene stimulated blood
mononuclear cell (MNC) apoptosis and receptor expression.
/IL-2 , FAS/

| | | | | Mitogene |
|---|---|---|---|---|
| | | PLB | Mitogene | stimulated + |
| | | incubated | stimulated for | PLB |
| | Control | 24 hour | 24 hour | 24 hour |
| Apoptosis | 2.2 | 3.0 | 2.5 | 3.8 |
| (% of total | | | | |
| cells) | | | | |
| IL-2 receptor | 3.8 | 2.9 | 33.4 | 1.2 |
| (%of total | | | | |
| cells) | | | | |
| Fas (CD95) | 46.3 | 10.5 | 22.5 | 6.2 |
| (% of total | | | | |
| cells) | | | | |

So under hypoxia the AAM activated aerobic glycolysis and decreased apoptosis. In aerobic conditions it enhanced the velocity aerobic glycolysis, but activated apoptosis and blocked expression of growth factors.

Apoptosis is very closely associated with growth-promoting ability of oncogenes. For example, potent antiapoptotic mithochondrial protein bcl-2 has growth inhibitory properties and Ras proteins the key transducers of mitogenic signals in normal and transformed cells trigger apoptosis (Kauffmann-Zeh, et al., 1997).

### Conclusion

These experiments demonstrate that effect of AMM sharply depended on the presence of oxygen. Under hypoxic conditions AAM acts as effective activator of the cell's energetic and blocator of apoptosis. But in aerobic conditions the AAM induced the opposite effect—stimulation of apoptosis and inhibition of the receptor expression induced by proliferation stimuli.

### References

1. Bakhutashvili V, Shakarishvili R, Geladze T, Tatishvili N, Bakhutashvili A, Cheishvili N, Chikovani T, Sobera LA. Drugs of the Future 1999; 24(9): 974-977.

2. Bakhutashvili A, Chikovani T, Cheishvili N, Bakhutashvili V. Immunomodilatory activity of Plaferon-LB. Georg Symp Project Development and Conversion; Collection of Reports 1995; 189-191.

3. Bakhutashvili V, Chikovani T, Bakhutashvili I, Cheishvili N, Kukuladze N, Bakhutashvili A. Some pharmacological characteristics of Immunomodulator Plaferon-LB. 10th International Congress of Immunology in New Delhi, The Immunologist 1998 Nov 1-6; 586.

4. Bakhutashvili V, Javakhishvili N, Tsagareli Z, Kipshidze N. Cardioprotective effects of Plaferon LB in a canine model. The J Heart Failure 1997 May; 4(1): 38, Abstr. #151.

5. Bakhutashvili V, Gagua M, Garishvili T, Gelashvili L, Kharebava G, Kvaratskhelia E. Human placenta antioxidant compounds of peptide nature. 11th International Symposium on Atherosclerosis, Paris 1997 October, Book of Abstracts; Abstract #1 and #2: 199.

6. Bakhutashvili V, Aladashvili A, Bakradze I. Impact of perioperational immunotherapy upon nonspecific resistance and specific immune status in patients with bowel cancer and upon frequency and spectrum of complications in nearest postoperative period. Int J Immunorehab 2000;2 (2): 4. Abstract #3.

7. Bakhutashvili V, Malashkhia V, Beridze M, Mikeladze D, Chikhladze M, Malashkhia J, Bakhutashvili A. Impact of Plaferon-LB upon drug-resistant forms of epilepsy. Int J Immunorehab 1996; 6: 32-37.

8. Bakhutashvili V, Telia D, Katsarava V. Use of Plaferon-LB and chromglycate in patients with symptomatic asthma. Int J Immunorehab 1999; 11: 214-215.

9. Bakhutashvili V, Pagava K, Telia A, Jorjoladze N, Kokaia L, Alavidze M, Kvachadze L.

Bronchotropic effect of Plaferon-LB. Int J Immunorehabilitation 1999; 11: 51-53.

10. Bakhutashvili V, Chikovani T, Bakhutashvili A, Dolidze T, Nanava N, Kvitaishvili G. Dynamics of some immunological indices in case of Plaferon-LB therapy. Europ. J. Allergy and Clin Immun 1999; 54(52 S): 77. Abstract #P37.

11. Bakhutashvili, A V, Jaguzhinsky, L S, Bakhutashvili, I V, Kadagidze, Z G, Baryshnikov, A Y, Sokolovskaya, A A, Zabotina, T N, Bakhutashvili, V I, “Amnion apoptosis modulator (“PLB”),” Int. J. of Immunorehab. 2001, 3(2): 17-22.

12. Beridze M, Bakhutashvili V. Impact of Plaferon-LB upon clinical and immunity indices in patients with ischemic stroke. Georgian Medical News 1998; 6(39): 27-30.

13. Beridze M, Bakhutashvili V, Malashkhia I. Plaferon-LB in therapy of patients with ischemic stroke. Int J Immunorehab 1999; 12: 27.

14. Beridze M, Bakhutashvili V, Malashkhia I. Plaferon-LB in therapy of patients with ischemic stroke. Int J of Immunorehab 1999; 12: 161-165.

15. Chavchanidze D, Hvadagiani G, Kalmahelidze V, Sulhanishvili V, Steptina J, Bahutashvili V, Managadze L. Protective effect of Plaferon LB-1 preparation on acute ischemic renal injury in experiments. Archivum Urologium Belgrade 1989; 30: 45-51.

16. Chavchanidze D, Sanikidze T, Bakhutashvili V, Managadze L. Changes of blood paramagnetic centers under the influence of shock waves on kidneys and membrane-protector effects of Plaferon-LB in experiment. Bulletin of the Georgian Academy of Sciences 1998; 158(2): 332-335.

17. Chavchanidze D, Sanikidze T, Bakhutashvili V, Managadze L. Determination of traumatic influence of shock waves and membrane-protecting effects of Plaferon-LB on the renal parenchyma during experimental lithotripsy.

Exper. Biology and Medicine 1998; 24(1-3): 65-72.

18. Chikovani T, Bakhutashvili A, Cheishvili N, Bakhutashvili V. Plaferon- LB: a new immunodilatory drug. Europ J Allergy Clin Immunol 1995; 50(6): 9.

19. Chikovani T, Bakhutashvili A, Bakhutashvili V, Imedidze E. Immunopharmacology of Plaferon-LB, Intern J Immunorehab 1994; 1: 44.

20. Chikovani T, Cheishvili N, Bakhutashvili A, Bakhutashvili V. The influence of Plaferon LB on synthesis of interleukins. Int J Immunorehab 1996; 3: 67.

21. Chikovani T, Cheishvili N, Pantsulaia I, Bakhutashvili V. Immuno-potential activity of fractions of Plaferon LB. Int J Immunorehab 1998; (9): 6.

22. Chikovani T, Cheishvili N, Pantsulaia I, Bakhutashvili A, Bakhutashvili V. Immuno-potential activity of Plaferon LB fractions. Int J Immunorehab 1998; (8): 249.

23. Chikovani T, Cheishvili N, Pantsulaia I, Bakhutashvili V, Bakhutashvili A. Immunologic study of human amniotic factor. Eur J Allergy Clin Immun; 1999; 54 (S 552): 89. Abstract #NP-81.

24. Chikovani T, Cheishvili N, Pirtskalava T, Pantsulaia I, Bakhutashvili V. Plaferon-LB as a blocker of neurotoxic effect of glutamate. Int. J Immunorehab 1997; 5:69.

25. Chikovani T, Rukhadze R, Sanikidze T, Pantsulaia I, Bakhutashvili V. Antioxidant effect of immunomodulator Plaferon LB in experimental thyroid pathology. Int J Immunorehab 1999; 12(S): 14-18.

26. Chikovani T, Bakhutasvili V, Cheishvili N, Kukuladze N. Several pharmacological features of Immunomodulator Plaferon LB, 10th International Congress of Immunology in New Delhi, The Immunologist 1998 Nov 1-6; 218.

27. Chikovani T, Bakhutasvili V, Rukhadze R. The perspectives of a new Immunomodulator Plaferon LB in endocrynologic practice. Proceedings of Tbilisi State Medical University 1996; 32: 21-23.

28. Chikovani T, Cheishvili N. Bakhutashvili V, Bakhutashvili A.Impact of Plaferon-LB upon the synthesis of interleukins. Int J Immunorehab 1996; 2: 71.

29. Dolidze C, Gelovani M, Bakhutashvili V, Chikovani T. Antirelapse effect in case of idiopatic nephropathy syndrome in children. Bull Acad Sci Geo 1992; 1: 209-11.

30. Gagua M, Dzidziguri D, Bakhutashvili V. Mechanisms of action of Plaferon-LB. Proceedings of The First Caucasus Simposium on Medical and Biological Sciences. Tbilisi-1999; 43-44.

31. Gagua M, Dzidziguri D. Influence of Plaferon LB on the transcriptional activity of regenerating liver and kidney cells. Int European A.I.R.R. Conference Tbilisi, 1999 October 4-9; Collection of reports: 11.

32. Gagua M, Dzidziguri D, Mikadze E, Bakhutashvili V. Influence of Plaferon-LB on morpho-functional activity of hepatocytes in white rats. Bulletin of Acad of Sci of Georgia 1999; 3: 56-68.

33. Gagua M, Russia L, Kupatadze R, Simonidze M, Bakhutashvili V. Investigation of polipeptide contents of Plaferon LB. Bulletin of Acad of Sci of Georgia 1996; 3(153): 450-452.

34. Gurgenidze G, Bakhutashvili V, Chogovadze M, Effect of Plaferon-LB in patients with steroid-resistant bronchial asthma. Eur J Allergy and Clin Immun 1999; 54 (52 S):77. Abstract #P532.

35. Johnson DW, Kipshidze N, Javahishvili N, Zagareli Z, Bakhutashvili V. Cardioprotective effects of Plaferon-LB in canine model. First International Congress on Heart Disease, Washington D.C., USA 1999 May 16-19; Book of Abstracts: 46. Abstract #30.

36. Kukuladze N, Cheishvili N, Bakhutashvili V, Bakhutashvili A. Effect of Plaferon-LB on proliferative activity of human periferal blood mononuclear cells and murine splenocytes. Reports of Georgian Academy of Science 1993; 148: 1.

37. Kokaia L, Telia A, Bakhutashvili V, Alavidze M.

Study of preventive effect of Plaferon-LB in guinea pigs with urgent bronchial reaction caused by inhalation of hystamin. Pediatrics at the Verge of XXI Century. Proceedings of Tbilisi State Medical Academy; Tbilisi 1999; Collection of reports: 147-149.

38. Macharadze D, Bakhutashvili V, Fedotova E. Plaferon-LB in immuno-rehabilitation of chidren with bronchial asthma. Int J Immunorehab 1998; 8: 249.

39. Maisuradze E, Garishvili T, Bakhutashvili V. Inhibition of Bee Venom Phospolipase A2 activity by Plaferon LB. Bulletin of the Georgian Academy of Sciences 1998; 2: 157.

40. Malashhia Y, Sepiashvili R, Nadareishvili Z, Vadachkoria L. Treatment of herpes zoster ganglioneuropathy with Plaferon. 5th Int World Cong Inflamm Antirheum Analg Immunomodul Geneva 1993 April 25-28; Abstract #301.

41. Metreveli D, Bakhutashvili A, Chikovani T, Pavliashvili D, Bakhutashvili V. Impact of Plaferon-LB on methabolic changes in blood of patients with B hepatitis. Georgian Medical News 1999; 9: 24-2.

42. Metreveli D, Bakhutashvili A, Chikovani T, Pavliashvili D, Bakhutashvili V. long term results of treatment of B hepatitis with Plaferon-LB. Int J Immunoreha 1999; 12: 48.

43. Metreveli D, Bakhutashvili V, Bochorishvili T, Jamutashvili M, Gingolava M. Influence of Plaferon-LB on clinical course of Hepatitis and Laboratory Data in Children. New Aspects in Hepatology and Gastroenterology. Falk Symposium 1998 May 29-30; Tbilisi (Georgia) Collection of reports: 195.

44. Metreveli D, Bakhutashvili V, Chikovani T, Bochorishvili T, Pavliashvili D. Results of treatment of acute B hepatitis by Plaferon LB. Bulletin of Georgian Academy of Science, Biology Series 1999 25 (1-3): 67-69.

45. Metreveli D, Chikovani T, Bakhutashvili V, Bakhutashvili A, Bochorishvili T, Kvitaishvili G, Jamutashvili M. Immunotherapy of patients with acute B hepatitis by Plaferon-LB. Clinical Immunology 1999; 90(3): 431. Abstract #83.

46. Metreveli D, Chikovani T, Bakhutashvili V, Bochorishvili T. New method of utilization of Plaferon -LB. Int J Immunorehab 1999; 12: 44.

47. Mikeladze D, Djanashia N, Djanashvili C, Bakhutashvili V. Influence of Plaferon upon main excitatory and inhibitary neurotransmitters of brain. Tbilisi 1995; 332-337.

48. Nadareishvili Z, Malashkhia Y, Bakhutashvili V. Plaferon in the treatment of herpes zoster ganglioneuritis in intravenous drug users. 9th int Conf AIDS. 4th STD World Cong Berlin 1993 June 6-11; 344. Abstract #PO-B08-122254.

49. Pantsulaia I, Cheishvili N, Kukuladze N, Jgenti M, Chikovani T. Influence of PlaferonLB on proliferative activity of splenocytes in iexperimental hyper- and hypothyroidism. Int J Immunorehab 2 (2): 49. Abstract #157.

50. Pantsulaia I, Pkhakadze E, Cheishvili N, Chikovani T, Jgenti M. Influence of Plaferon LB on the course of moderate periodontitis. Int J Immunorehabilitation 2000; 2 (2): 89. Abstract #294.

51. Pantsulaia I, Chikovani T, Cheishvili N, Garishvili T, Kharebava G, Bakhutashvili V. Impact of Plaferon-LB on functional activity of lymphocytes. Bulletin of Georgian Academy of Science Biology Series 1999; 3: 1-3.

52. Pantsulaia I, Chikovani T, Ruhadze R, Sanikidze T, Bakhutashvili V.The impact of Plaferon-LB on changes in immune organs caused by acute experimental hyperthyroidism. Proceedings of the 4th National Scientific Conference, Kutaisi, 1998 May 31; Collection of reports: 24. of Georgia, TRANSACTIONS, I, Tbilisi, 1999, P.1-5-18

53. Pantsulaia I, Chikovani T, Rukhadze R, Sanikidze T, Bakhutashvili V. Influence of Plaferon LB on some morphometric indices of spleen during experimental hyper- and hypothyroidism. Int J.Immunorehab 1999; 14: 117

54. Pantsulaia I, Chikovani T, Rukhadze R, Sanikidze T, Bakhutashvili V. Influence of Plaferon LB on nitric oxide metabolism during thyrotoxikosis. Bulletin of the Georgian Academy of Sciences 1999; 160 (3): 571-573.

55. Pavliashvili D, Chikovani T, Metreveli D, Sanikidze T, Bakhutashvili V. Influence of sublingual administration of Plaferon-LB on metabolic disorders in viral B hepatitis. Georgian Med News 1999; 9: 54.

56. Ruhadze P, Ciqovani T, Bakhutashvili V, Sanikidze T, Metreveli D, Balarjishvili M. The impact of Plaferon LB on the metabolism of nitric oxide in thyrotoxicosis. Bulletin of Georgian Academy of Science 1999; 160: 3.

57. Ruhadze R, Chikovani T, Bakhutashvili V, Sanikidze T, Metreveli D, Balarjishvili M. The impact of Plaferon LB on the metabolism of nitric oxide in hypothyreosis. Bulletin of Georgian Academy of Science; 2000, 161:1.

58. Ruhadze R, Sanikidze T, Ciqovani T, Pantsulaia I, Bakhutashvili V, Characteristics of blood paramagnetic centers in experimental hyperthyroidism. Bulletin of Georgian Academy of Science 1998; (4-5-6): 45-47.

59. Ruhadze R, Nicolaishvili L, Bakhutashvili V. The impact of Plaferon LB on several morphometric indices of thyroid gland in L-Thyroxine hyperthyroidosis. Int J Immunorehab 1996; 2: 152.

60. Ruhadze R, Sanikidze T, Chikovani T, Pantsulaia I, Bakhutashvili V, Nicolaishvili L. The impact of Plaferon LB on changes in liver metabolic paramagnetic centers under the conditions of experimental hyperthyroidosis. Bulletin of Georgian Academy of Science 1998; 1-3: 91-95.

61. Ruhadze R, Sanikidze T., Chikovani T, Pantsulaia I, Bakhutashvili V, Jgenti M. The impact of Plaferon LB on metabolic changes in myocardium under the conditions of experimental hyperthyroidosis. Bulletin of Georgian Academy of Science 1998; 1-3: 95-98.

62. Ruhadze R, Sanikidze T, Ciqovani T, Bakhutashvili V. Impact of Plaferon LB on structural and functional changes in mitochondria of hepatocites under the conditions of experimental hyperthyroidosis. Georgian Medical News 1999; 2: 3-5.

63. Rjazantzeva S, Visotskaja I, Ermilova V, Bakhutashvili V. Morphologic and immune characteristics of breast cancer after preoperative use of immunemodulator Plaferon LB. Bulletin of Oncology National Centre, Russian Academy of Science 1998; 4: 37.

64. Tatishvili N, Bakhutashvili V, Simonia G. Local use of Plaferon LB in rheumathology. Int J Immunorehab 1999; 12: 81.

65. Telia À, Bakhutashvili V, Jorjoladze N. Use of Plaferon LB in children with bronchial asthma. Int J Immunorehab 1997; 7: 139.

66. Telia À, Bakhutashvili V, Kokaia L, Jorjoladze N, Alavidze M, Kvachadze L. Comparison of effect of Plaferon LB and chromglycate in adults with bronchial asthma. Int J Immunorehab 1999; 12: 164-165.

67. Telia À, Bakhutashvili V, Kokaia L, Jorjoladze N, Kvachadze L. Preventive effect of Plaferon LB on hystamine-caused bronchial obstruction. Int J Immunorehab 1998; 8: 125.

68. Telia À, Bakhutashvili V, Kokaia L,Alavidze M, Pagava K, Jorjoladze N, Kvachadze L. Plaferon LB as an alternative treatment of bronchial asthma in children. Int J Immunorehab 1998; 10: 165-167.

69. Telia À, Katsarava V, Bakhutashvili V. Impact of Plaferon LB on bronchial and alveolar cell count and parameters of external respiration. Int J Immunorehab 1999; 12: 24.

70. Bakhutashvili A, Jaguzhinsky L, Bakhutashvili I, Kadagidze D, Baryshnikov A, Sokolovskaya A, Zabotina T, Bakhutashvili V, Amnion apoptosis modulator. Int J Immunorehab 2001; 3(2): 17-22.

71. Ashkenazi A, Dixit V M, Science 1999, 281, 1305. 72. Beraud C, Henzel W J, Baeuerle P A, Proc Natl Acad Sci. 1999, 96, 429.

73. Blancher C, Harris A L, Cancer Metastasis Rev 1998, 17, 2, 187.

74. Campbell I K, Gerondakis S, O'Donnell K, Wicks I P, J Clin Invst 2000,105, 1799.

75. Carmeliet P, Dor Y, Herbert J M et al. Nature, 1998, 394, 485.

76. Ema M, Taya S, Yokotani N, Sogawa K, Matsuda Y, Fjuii-Kuriyama Y, Proc.Natl Acad Sci, 1997, 94, 4273-4278.

77. Evan G, Littlewood T, Science, 1998, 281, 13. 78. Feldman M, Brennan F M, Maini R N, Cell, 1996, 85, 307.

79. Guillemin K, Krasnow M A, Cell1997, 89, 9.

80. Hellwig-Burgel T H, Rutkovski K, Metzen E, Fandrey J, Jelkmann W, Blood 1999, 94, 5, 1561

81. Jacobson M D, Weil M, Raff M C, Cell 1997, 88, 347.

82. Imbert V, et al. Cell 1996, 86, 787.

83. Kauffmann-Zeh A. et al, Nature 1997, 385, 544.

84. Koch AE Arthritis Rheum. 1998, 41, 951.

85. Kong Y et al, Nature 1999, 402, 304.

86. Nicoletti I., Migliorati G., Pagliacci M. C., et al. J Immunol. Methods; 1991. 139: 271-80.

87. Ratclif P J, O'Rourke J F, Maxwell P H, Pugh C W, J Exp Biol 1998, 201(Pt 8), 1153

88. Rothwarf D M and Karin M, Science STKE (1999), Reviews, www.stke.org/cgi/content/full/OC sigtranc;1999/5/re1

89. Schmedtje J F et al., J. Biol. Chem. 1997, 272, 601-608.

90. Steller H, Science 1995, 267, 1445.

91. Tian H, McKnight S L, Russell D W, Genes Dev. 1997 11, 72-82.

92. Thatte U, Dahanukar S, Drugs 1997, 54, 4, 511.

93. Yan SF et al. J Biol Chem 1995, 270, 11463-11471.

94. Wenger R H, Gassmann M, Biol Chem. 1997, 378, 609.

95. Wang G L, Jiang B -H, Rue EA., Semenza G L. Proc. Natl. Acad. Sci. USA, (1995) 92,5510-5514.

96. Nakashidze I, Rukhadze R, Chikovani T, Bakhutashvili V, “The influence of Plaferon LB on the liver and myocardium of white rats in the experimental traumatic shock,” Proceedings of VII International Congress on Immunorehabilitation “Allergy, Immunology and Global Network: Insight into the new Millenium,” New York, NY, USA, April 14-17, 2001, Int. J. Immunorehab. 3 (2): 89, Abstr #120

What is claimed is:

1. Amniotic apoptosis modulating substances.

2. A composition with apoptosis modulating activity obtainable from human amniotic tissue prepared according to a method comprising the steps of:
priming, induction, biosynthesis, and purification of the product.

3. A composition of claim 2 including lyophilizing the product.

4. A composition with apoptosis modulating activity obtainable from human amniotic tissue prepared according to a method comprising the steps of:
extraction and purification of the product.

5. The composition according to claim 2, 3 and 4 further comprising a pharmaceutically effective agent.

6. The composition according to claim 5, wherein the agent is selected from the group consisting of antibiotics, wound healing agents, antioxidants, antivirals, antifungals, anti-ischemics, anti-injury, and anti-aging, immunomodulatory, anti-hypoxic, anti-toxic, anti-allergic, anti-wrinkle, anti-inflammatory, anti-infectious, anti-immunogenic, and anti-neoplastic.

7. The composition according to claim 2, 3 and 4, further comprising a physiologically acceptable carrier.

8. The composition according to claim 7 wherein the carrier is suitable for topical administration.

9. The composition according to claim 7 wherein the carrier is suitable for parenteral administration.

10. The composition according to claim 7 wherein the carrier is suitable for gastrointestinal administration.

11. A composition with apoptosis modulating activity derived from human amniotic tissue, amnion tissue products or tissue.

12. A composition with apoptosis modulating activity produced chemically or by using genetic engineering or by synthesis.

13. The composition according to claim 11 and 12, further comprising a pharmaceutically effective agent.

14. The composition according to claim 13, wherein the agent is selected from the group consisting antibiotics, wound healing agents, antioxidants, antivirals, antifungals, anti-ischemics, anti-injury, anti-aging, immunomodulatory, anti-hypoxic, anti-toxic, anti-allergic, anti-wrinkle, anti-inflammatory, anti-infectious, anti-immunogenic, and anti-neoplastic.

15. The composition according to claim 13 and 14, further comprising a physiologically acceptable carrier.

16. The composition according to claim 15 wherein the carrier is suitable for topical administration.

17. The composition according to claim 15 wherein the carrier is suitable for parenteral administration.

18. The composition according to claim 15 wherein the carrier is suitable for gastrointestinal administration.

19. A method for producing a composition with derived from human amniotic tissue, amnion tissue products or tissue activity obtainable from human amniotic tissue comprising the steps of:
priming,
induction,
biosynthesis, and
purification.

20. A method of claim 19 including lyophilizing.

21. A method for producing a composition with apoptosis modulating activity obtainable from human amniotic tissue comprising the steps of:
extraction and purification.

22. A method for producing a composition derived from human amniotic tissue, amnion tissue products or tissue activity obtainable from amniotic tissue and/or prepared from chemical formulation, genetic engineering or synthesis.

23. A method for improving the skin condition of a subject comprising contacting an effective amount of a composition with anti-apoptotic, anti-wrinkle, anti-aging, or anti-drying activity obtainable from human amniotic tissue with said skin surface on the subject.

24. A method of claim 23 wherein the composition with anti-apoptotic, anti-wrinkle, anti-aging, or anti-drying activity is prepared from chemical formulation, genetic engineering or synthesis.

25. A method for normalizing the biochemical parameters of liver function and immunologic indices in an acute viral hepatitis B subject, speeding recovery from symptoms of the disease, and preventing recurrence of the disease with an apoptosis modulating composition obtainable from human amniotic tissue by administering an effective amount of the composition to the subject.

26. A method of claim 25 wherein the composition with apoptosis modulating activity is prepared from chemical formulation, genetic engineering or synthesis.

27. A method of normalizing cell counts of CD3+, CD4+, CD8+, and T-cells carrying HLA-DR antigens and improving neurological symptoms in a herpes zoster ganglioneuritis subject with an anti-apoptotic composition obtainable from human amniotic tissue by administering an effective amount of the composition to the subject.

28. A method of claim 27 wherein the composition with apoptosis modulating activity is prepared from chemical formulation, genetic engineering or synthesis.

29. A method of normalizing levels of CD3+ and CD4+ T-cell phenotypes in a diabetic peripheral polyneuropathy subject with an apoptosis modulating obtainable from human amniotic tissue by administering an effective amount of the composition to the subject.

30. A method of claim 29 wherein the composition with apoptosis modulating activity is prepared from chemical formulation, genetic engineering or synthesis.

31. A method of promoting earlier and prolonged clinical laboratory remission in a child with Idiopathic Nephropathy Syndrome (INS) and correcting the reduction in CD3+ and CDB+ T lymphocytes in the same subject with an apoptosis modulating composition obtainable from human amniotic tissue combined with prednisolone by administering an effective amount of the composition to the subject.

32. A method of claim 31 wherein the composition with apoptosis modulating activity is prepared from chemical formulation, genetic engineering or synthesis.

33. A method of improving clinical symptoms and laboratory indices, stimulating leukocyte interferon-genesis and normalizing humoral and cellular immunity in a juvenile rheumatoid arthritis, rheumatoid arthritis or psoriatic arthritis subject with an apoptosis modulating composition obtainable from human amniotic tissue by administering an effective amount of the composition to the subject.

34. A method of claim 33 wherein the composition with apoptosis modulating activity is prepared from chemical formulation, genetic engineering or synthesis.

35. A method of reducing the average daily dose of oral steroid required for relief;
moderately improving spirometric parameters; and
increasing sensitivity to dexamethasone in a bronchial asthma subject with an apoptosis modulating composition obtainable from human amniotic tissue by administering an effective amount of the composition to the subject.

36. A method of claim 35 wherein the composition with apoptosis modulating activity is prepared from chemical formulation, genetic engineering or synthesis.

37. A method of improving immunological indices and decreasing the frequency of infections in a pediatric patient with respiratory infection with an apoptosis modulating composition obtainable from human amniotic tissue by administering an effective amount of the composition to the subject.

38. A method of claim 37 wherein the composition with apoptosis modulating activity is prepared from chemical formulation, genetic engineering or synthesis.

39. A method of reducing allergic reactions and drug toxicity in an epileptic subject who uses anticonvulsants with an apoptosis modulating composition obtainable from human amniotic tissue by administering an effective amount of the composition to the subject.

40. A method of claim 39 wherein the composition with apoptosis modulating activity is prepared from chemical formulation, genetic engineering or synthesis.

41. A method of immunomodulation, normalizing of levels of the tumor serum marker, CA15.3, and increasing tumor-infiltrating CD5′ T-cells and CD11 macrophages in an early breast cancer subject with an apoptosis modulating composition obtainable from human amniotic tissue by administering an effective amount of the composition to the subject.

42. A method of claim 41 wherein the composition with apoptosis modulating activity prepared from chemical formulation, genetic engineering or synthesis.

43. A method of improving clinical symptoms, eradicating rash, relieving pain, increasing activity of immunoregulatory lymphocytes and percentages of CD3+ and CD8 in a psoriasis subject with an apoptosis modulating composition obtainable from human amniotic tissue by administering an effective amount of the composition to the subject.

44. A method of claim 43 wherein the composition with apoptosis modulating activity is prepared from chemical formulation, genetic engineering or synthesis.

45. A method of treating atherososclerotic and other forms of vascular obstructions that cause ischemia of the myocardium and other tissues in a human subject with an apoptosis modulating composition obtainable from human amniotic tissue by administering an effective amount of the composition to the subject.

46. A method of claim 45 wherein the composition with apoptosis modulating activity is prepared and/or prepared from chemical formulation, genetic engineering or synthesis.

47. A method of limiting myocardial cell death due to viral and immunogenic myocardiopathies with an apoptosis modulating composition obtainable from human amniotic tissue by administering an effective amount of the composition to the subject.

48. A method of claim 47 wherein the composition with apoptosis modulating activity is prepared from chemical formulation, genetic engineering or synthesis.

49. A method of limiting the rejection reaction that follows organ transplantation with an apoptosis modulating composition obtainable from human amniotic tissue by administering an effective amount of the composition to the subject.

50. A method of claim 49 wherein the composition with apoptosis modulating activity is prepared from chemical formulation, genetic engineering or synthesis.

51. A method of treating HIV infection with an apoptosis modulating composition obtainable from human amniotic tissue by administering an effective amount of the composition to the subject.

52. A method of claim 51 wherein the composition with apoptosis modulating activity is prepared from chemical formulation, genetic engineering or synthesis.

53. A method of treating brain ischemia and trauma with an apoptosis modulating composition obtainable from human amniotic tissue by administering an effective amount of the composition to the subject.

54. A method of claim 53 wherein the composition with apoptosis modulating activity is prepared from chemical formulation, genetic engineering or synthesis.

55. A method of treating the pathologic consequences of ischemia-reperfusion with an apoptosis modulating composition obtainable from human amniotic tissue by administering an effective amount of the composition to the subject.

56. A method of claim 55 wherein the composition with apoptosis modulating activity is prepared from chemical formulation, genetic engineering or synthesis.

57. A method of treating alcohol and morphine intoxication with an apoptosis modulating composition obtainable from human amniotic tissue by administering an effective amount of the composition to the subject.

58. A method of claim 57 wherein the composition with apoptosis modulating activity is prepared from chemical formulation, genetic engineering or synthesis.

59. A method of treating wound healing with an apoptosis modulating composition obtainable from human amniotic tissue by administering an effective amount of the composition to the subject.

60. A method of claim 59 wherein the composition with apoptosis modulating activity is prepared from chemical formulation, genetic engineering or synthesis.

61. A method of treating viral diseases with an apoptosis modulating composition obtainable from human amniotic tissue by administering an effective amount of the composition to the subject.

62. A method of claim 61 wherein the composition with apoptosis modulating activity is prepared from chemical formulation, genetic engineering or synthesis.

63. A composition with apoptosis modulating activity obtainable from human amniotic tissue having characteristic peaks as set forth in FIG. 2.

64. A composition with apoptosis modulating activity obtainable from human amniotic tissue having at least one characteristic peak as set forth in FIG. 2.

65. A composition with apoptosis modulating activity obtainable from human amniotic tissue having characteristic peaks as set forth in FIG. 3.

66. A composition with apoptosis modulating activity obtainable from human amniotic tissue having at least one characteristic peak as set forth in FIG. 3.

67. A method for protecting cardiomyocytes from injury, comprising contacting said cardiomyocytes with an effective amount of the composition of claim 65.

68. A method for protecting cardiomyocytes in a subject comprising administering to the subject an effective amount of the composition of claim 65 to said subject.

69. The method of claim 67 or 68, wherein the cardiomyocyte is chemically injured.

70. A composition capable of inhibiting or killing cancer cells, wherein said composition is obtainable from human amniotic tissue with apoptosis modulating activity.

71. A method of inhibiting or killing cancer cells comprising contacting said cancer cells with an effective amount of claim 70.

72. A method of inhibiting or killing cancer cells comprising administering to the subject an effective amount of the composition of claim 68 to said subject.

73. A composition which is antagonistic to H1-histamine receptor, wherein said composition is obtainable from human amniotic tissue with apoptosis modulating activity.

74. A method to produce effects which are antagonistic to H1-histamine receptor in a cell, comprising contacting said cell with an effective amount of the composition of claim 73.

75. A method to produce effects which are antagonistic to H1-histamine receptor in a subject comprising administering to the subject an effective amount of the composition of claim 73 to said subject.

76. A composition which is inhibitory to A2-phospholipase activity, wherein said composition is obtainable from human amniotic tissue with apoptosis modulating activity.

77. A method for producing inhibitory A2-phospholipase activity in a cell comprising contacting said cells with an effective amount of the composition of claim 76.

78. A method for producing inhibitory A2-phospholipase activity in a subject comprising administering to the subject an effective amount of the composition of claim 76 to said subject.

79. A composition for protecting cardiomyocytes, wherein said composition is obtainable from human amniotic tissue with apoptosis modulating activity.

80. A method for protecting cardiomyocytes in a cell comprising contacting said cell with an effective amount of the composition of claim 79.

81. A method for protecting cardiomyocytes in a subject comprising administering to the subject an effective amount of the composition of claim 79 to said subject.

82. A composition for protecting against the effects of Tumor Necrosis Factor (TNF), wherein said composition is obtainable from human amniotic tissue with apoptosis modulating activity.

83. A method for protecting against the effects of Tumor Necrosis Factor (TNF) in a cell comprising contacting said cell with an effective amount of the composition of claim 82.

84. A method for protecting against the effects of Tumor Necrosis Factor (TNF) in a subject comprising administering to the subject an effective amount of the composition of claim 82 to said subject.
